(19) United States
(12) Patent Application Publication
(10) Pub. No.: US 2010/0152466 A1
(43) Pub. Date: Jun. 17, 2010

(54) INTEGRATED METHOD FOR PRODUCING TRIOXANE FROM FORMALDEHYDE

(76) Inventors: Neven Lang, Mannheim (DE); Eckhard Stroefer, Mannheim (DE); Achim Stammer, Freinsheim (DE); Thorsten Friese, Mannheim (DE); Markus Siegert, Heidelberg (DE); Hans Hasse, Kaiserslautern (DE); Thomas Gruetzner, Brig (CH); Sergej Blagov, Stuttgart (DE)

Correspondence Address: CONNOLLY BOVE LODGE & HUTZ, LLP, P O BOX 2207, WILMINGTON, DE, 19899, US

(21) Appl. No.: 12/063,171

(22) PCT Filed: Aug. 4, 2006
(86) PCT No.: PCT/EP06/65089

§ 371 Date: Feb. 7, 2008

(30) Foreign Application Priority Data
Aug. 8, 2005 (DE) 10 2005 037 293.7

(51) Int. Cl. C07D 323/06

(52) U.S. Cl. 549/368

(57) ABSTRACT

The invention relates to an integrated process for preparing trioxane from formaldehyde in which a stream A1 comprising water and formaldehyde and a recycle stream B2 consisting substantially of water and formaldehyde are fed to a trioxane synthesis reactor in which the formaldehyde is converted to trioxane to obtain a product stream A2 comprising trioxane, water and formaldehyde; stream A2 is fed to a first distillation column and distilled at a pressure in the range from 0.1 to 2.5 bar to obtain a stream B1 enriched in trioxane, and the stream B2 consisting substantially of water and formaldehyde; stream B1 and a recycle stream D1 comprising trioxane, water and formaldehyde are fed to a second distillation column and distilled at a pressure in the range from 0.2 to 17.5 bar to obtain a product stream C2 consisting substantially of trioxane, and a stream C1 comprising trioxane, water and formaldehyde; stream C1 is fed to a third distillation column and distilled at a pressure in the range from 0.1 to 2.5 bar to obtain the recycle stream D1 comprising trioxane, water and formaldehyde, and a stream D2 consisting substantially of water and formaldehyde.

The invention relates to an integrated process for preparing trioxane from formaldehyde.

Trioxane is generally prepared by reactive distillation of aqueous formaldehyde solution in the presence of acidic catalysts. This affords a mixture comprising trioxane, formaldehyde and water as distillate. The trioxane is subsequently extracted from this mixture by extraction with halogenated hydrocarbons such as methylene chloride or 1,2-dichloroethane, or other water-immiscible solvents.

DE-A 1 668 867 describes a process for removing trioxane from mixtures comprising water, formaldehyde and trioxane by extraction with an organic solvent. In this process, an extraction zone consisting of two subzones is charged at one end with an organic, virtually water-immiscible extractant for trioxane, and at the other end with water. Between the two subzones, the distillate from the trioxane synthesis to be separated is fed. On the side of the solvent feed, an aqueous formaldehyde solution is then obtained, and, on the side of the water feed, a virtually formaldehyde-free solution of trioxane in the solvent.

A disadvantage of this procedure is the occurrence of extractant which has to be purified. Some of the extractants used are hazardous substances (T or T⁺ substances in the context of the German Hazardous Substances Directive), whose handling entails special precautions.

DE-A 197 32 291 describes a process for removing trioxane from an aqueous mixture which consists substantially of trioxane, water and formaldehyde, by removing trioxane from the mixture by pervaporation and separating the trioxane-enriched permeate by rectification into pure trioxane on the one hand and an azeotropic mixture of trioxane, water and formaldehyde on the other. In one example, an aqueous mixture consisting of 40% by weight of trioxane, 40% by weight of water and 20% by weight of formaldehyde is separated in a first distillation column under standard pressure into a water/formaldehyde mixture and into an azeotropic trioxane/water/formaldehyde mixture. The azeotropic mixture is passed into a pervaporation unit which comprises a membrane composed of polydimethylsiloxane with a hydrophobic zeolite. The trioxane-enriched mixture is separated in a second distillation column under standard pressure into trioxane and, in turn, into an azeotropic mixture of trioxane, water and formaldehyde. This azeotropic mixture is recycled upstream of the pervaporation stage.

This procedure is very costly and inconvenient. The pervaporation unit in particular entails high capital costs.

It is an object of the invention to provide an alternative process for preparing trioxane from aqueous formaldehyde solution to obtain pure trioxane. It is a particular object to provide a process which avoids the performance of extraction steps or pervaporation steps for obtaining pure trioxane.

The object is achieved by an integrated process for preparing trioxane from formaldehyde, which comprises the following steps:
a) a stream A1 comprising water and formaldehyde and a recycle stream B2 consisting substantially of water and formaldehyde are fed to a trioxane synthesis reactor in which the formaldehyde is converted to trioxane to obtain a product stream A2 comprising trioxane, water and formaldehyde;
b) stream A2 is fed to a first distillation column and distilled at a pressure in the range from 0.1 to 2.5 bar to obtain a stream B1 enriched in trioxane, and the stream B2 consisting substantially of water and formaldehyde;
c) stream B1 and a recycle stream D1 comprising trioxane, water and formaldehyde are fed to a second distillation column and distilled at a pressure in the range from 0.2 to 17.5 bar to obtain a product stream C2 consisting substantially of trioxane, and a stream C1 comprising trioxane, water and formaldehyde;
d) stream C1 is fed to a third distillation column and distilled at a pressure in the range from 0.1 to 2.5 bar to obtain the recycle stream D1 comprising trioxane, water and formaldehyde, and a stream D2 consisting substantially of water and formaldehyde.

Substantially consisting of one or more components means that these components are present to an extent of at least 90% by weight, preferably to an extent of at least 95% by weight, in the appropriate stream.

It is known that trioxane, formaldehyde and water form a ternary azeotrope which, at a pressure of 1 bar, has the composition of 69% by weight of trioxane, 5% by weight of formaldehyde and 26% by weight of water. According to the invention, the ternary azeotrope is separated by a pressure swing distillation, by carrying out a first and a second distillation stage at different pressures. In a first distillation stage which is operated at lower pressure, the starting mixture is separated into a trioxane-rich trioxane/water/formaldehyde mixture with low formaldehyde content on the one hand and a substantially trioxane-free formaldehyde/water mixture on the other. The trioxane-rich trioxane/water/formaldehyde mixture is subsequently separated in a second distillation stage which is carried out at high pressure into a trioxane-rich trioxane/water/formaldehyde mixture on the one hand and pure trioxane on the other. The trioxane-rich trioxane/water/formaldehyde mixture is subsequently separated in a second distillation stage which is carried out at high pressure into a trioxane-rich trioxane/water/formaldehyde mixture on the one hand and pure trioxane on the other. According to the invention, the trioxane-rich trioxane/water/formaldehyde mixture is fed to a third distillation stage which is preferably operated at the same pressure as the first distillation stage. In the third distillation stage, a substantially trioxane-free water/formaldehyde mixture and a trioxane/water/formaldehyde mixture are obtained. The trioxane/water/formaldehyde mixture is recycled into the second distillation stage. This achieves substantially all trioxane prepared in the synthesis being obtained as a product of value.

According to the invention, each distillation stage comprises a distillation column. Suitable distillation columns are any distillation columns such as columns with structured packing or random packing. The distillation columns may comprise any internals, structured packings or random packings. In the following, all pressure data relate to the pressure at the top of the column in question.

In a first process step a), a stream A1 comprising water and formaldehyde and a recycle stream B2 consisting substantially of water and formaldehyde are fed to a trioxane synthesis reactor and allowed to react to obtain a product stream A2 comprising trioxane, water and formaldehyde. The reaction is effected preferably under acidic conditions.

Streams A1 and B2 can be fed separately. However, it is also possible to mix streams A1 and B2 before they are fed into the trioxane synthesis reactor.

In general, stream A1 comprises from 50 to 85% by weight of formaldehyde and from 15 to 50% by weight of water.

The ratio of streams A1 and B2 is preferably selected such that, overall, from 15 to 70% by weight of water and from 30 to 85% by weight of formaldehyde, more preferably from 20 to 63% by weight of water and from 37 to 80% by weight of formaldehyde, are fed to the trioxane synthesis reactor.

Product stream A2 comprises generally from 35 to 84% by weight of formaldehyde, from 15 to 45% by weight of water and from 1 to 30% by weight of trioxane.

In one embodiment of the process according to the invention, the water/formaldehyde mixture is reacted in the trioxane synthesis stage a) in the presence of acidic homogeneous or heterogeneous catalysts such as ion exchange resins, zeolites, sulfuric acid and p-toluenesulfonic acid at a temperature of generally from 70 to 130° C. It is possible to work in a reactive distillation column or a reactive evaporator. The product mixture composed of trioxane, formaldehyde and water is then obtained as a vaporous vapor draw stream of the reactive evaporator or as a top draw stream of the reactive distillation column. The trioxane synthesis may also be carried out in a fixed bed reactor or fluidized bed reactor over a heterogeneous catalyst, for example an ion exchange resin or zeolite.

In a step b) which follows step a), stream A2 is fed to a first distillation column and distilled at a pressure of from 0.1 to 2.5 bar, preferably from 0.4 to 1.5 bar, for example 1 bar, to obtain a stream B1 enriched in trioxane, and the stream B2 consisting substantially of water and formaldehyde.

The first distillation column comprises preferably from 2 to 50, more preferably from 4 to 40 theoretical plates. In general, the stripping section of the first distillation column comprises at least 25% of the number of theoretical plates of the distillation column. The rectifying section preferably comprises from 50 to 90% of the theoretical plates of this distillation column.

The stream B1 enriched in trioxane comprises generally from 20 to 60% by weight of trioxane, from 15 to 79% by weight of water and from 1 to 25% by weight of formaldehyde. The trioxane-enriched stream B1 preferably comprises from 25 to 55% by weight of trioxane, from 25 to 70% by weight of water and from 5 to 20% by weight of formaldehyde. Stream B2 comprises generally from 51 to 85% by weight of formaldehyde, from 15 to 49% by weight of water and from 0 to 1% by weight of trioxane. Stream B2 preferably comprises less than 0.5% by weight of trioxane, more preferably less than 0.1% by weight of trioxane.

Stream A2 is fed to the first distillation column preferably in the bottom or as a side feed in the stripping section of the column. Stream B1 is withdrawn from the first distillation column preferably as a top draw stream and stream B2 as a bottom draw stream. Stream B1 may also be withdrawn as a side draw stream below the top of the column.

In a further embodiment of the process according to the invention, the trioxane synthesis stage a) and the first distillation stage b) are carried out together as a reactive distillation in a reaction column. In the stripping section, this may comprise a fixed catalyst bed of a heterogeneous catalyst. Alternatively, the reactive distillation may also be carried out in the presence of a homogeneous catalyst, in which case an acidic catalyst is present together with the water/formaldehyde mixture in the column bottom.

In a process step c) which follows step b), the trioxane-enriched stream B1 and a recycle stream D1 comprising trioxane, water and formaldehyde are fed to a second distillation column and distilled at a pressure of from 0.2 to 17.5 bar to obtain a stream C2 consisting substantially of pure trioxane, and a stream C1 comprising trioxane, water and formaldehyde.

The second distillation column comprises generally at least 2 theoretical plates, preferably from 10 to 50 theoretical plates. In general, the stripping section of this distillation column comprises from 25 to 90%, preferably from 50 to 75%, of the theoretical plates of this column.

The pressure in the second distillation column is at least 0.1 bar higher than in the first distillation column. In general, this pressure difference is from 0.5 to 10 bar, preferably from 1 to 7 bar. The second distillation column is operated preferably at a pressure between 2 and 10 bar, more preferably at a pressure between 2 and 7 bar.

Product stream C2 comprises generally from 95 to 100% by weight, preferably from 99 to 100% by weight of trioxane, and from 0 to 5% by weight, preferably from 0 to 1% by weight of water. More preferably, the content of water in the product stream is <0.1%. It may even be <0.01%. Stream C1 comprises, for example, from 5 to 20% by weight of formaldehyde, from 15 to 40% by weight of water and from 40 to 70% by weight of trioxane.

Preferably, stream B1 is fed as a first side feed and stream D1 as a second side feed above the first side feed to the second distillation column. It is also possible for streams B1 and D1 to be mixed before they are fed to the column. In this case, the feed is preferably a side feed.

Stream C1 is preferably withdrawn from the second distillation column as a top draw stream and product stream C2 as a bottom draw stream.

The ratio of streams B1 and D1 to one another is preferably selected such that, overall, from 1 to 25% by weight of formaldehyde, from 5 to 69% by weight of water and from 30 to 80% by weight of trioxane, preferably from 3 to 20% by weight of formaldehyde, from 5 to 57% by weight of water and from 40 to 75% by weight of trioxane are fed to the second distillation stage.

In the step d) which follows step c), stream C1 is fed to a third distillation column and distilled at a pressure in the range from 0.1 to 2.5 bar to obtain the recycle stream D1 comprising trioxane, water and formaldehyde and a product stream D2 consisting substantially of water and formaldehyde.

The pressure in the third distillation column is generally from 0.1 to 15 bar, preferably from 0.5 to 10 bar and in particular from 1 to 7 bar lower than the pressure in the second distillation column. In a preferred embodiment, the pressure in the third distillation column is in the range from 0.5 to 2.0 bar, more preferably in the range from 0.4 to 1.5 bar, and corresponds to the pressure in the first distillation column.

The third distillation column comprises generally at least 2 theoretical plates, preferably from 10 to 50 theoretical plates. In general, the stripping section of the third distillation column comprises from 25 to 90%, preferably from 50 to 75% of the theoretical plates of this column.

Stream D1 comprises generally from 50 to 80% by weight of trioxane, from 1 to 20% by weight of formaldehyde and from 5 to 49% by weight of water. Stream D1 comprises preferably from 55 to 75% by weight of trioxane, from 3 to 15% by weight of formaldehyde and from 10 to 42% by weight of water. Stream D2 comprises, for example, from 0 to 1% by weight of trioxane, from 10 to 50% by weight of formaldehyde and from 60 to 90% by weight of water. Stream D2 comprises preferably from 0 to 0.5% by weight of trioxane, from 15 to 40% by weight of formaldehyde and from 60 to 85% by weight of water.

In general, stream C1 is fed to the third distillation column as a side feed. Stream D1 is obtained generally as a top draw stream and stream D2 as a bottom draw stream or as a side draw stream in the stripping section of the column.

In a preferred embodiment, the process according to the invention additionally comprises the following steps:
e) a feed stream F1 comprising water and formaldehyde is fed to a formaldehyde concentration unit, stream A1 is withdrawn from the concentration unit as a formaldehyde-rich bottom draw stream and a low-formaldehyde stream F2 is withdrawn as a top or vapor draw stream
f) stream D2 and stream F2 are fed to a fourth distillation column and distilled at a pressure in the range from 1 to 10 bar to obtain a stream E1 comprising water and formaldehyde and a stream E2 consisting substantially of water.

Step e) precedes step a) and step f) follows step d).

In the step f) which follows step d), stream D2 and a stream F2 obtained in the formaldehyde concentration unit are fed to the fourth distillation column and distilled at a pressure in the range from 1 to 10 bar to obtain a stream E1 comprising water and formaldehyde and a stream E2 consisting substantially of water.

The fourth distillation stage is carried out preferably at a pressure between 2 and 7 bar.

The fourth distillation column has at least 2 theoretical plates, preferably from 10 to 50 theoretical plates. In general, the stripping section of this distillation column comprises from 25 to 90%, preferably from 30 to 75% of the theoretical plates of this column.

Stream E2 comprises generally at least 90% by weight, preferably at least 95% by weight and more preferably at least 97% by weight of water. Stream E1 comprises generally from 0 to 2% by weight of trioxane, from 40 to 80% by weight of formaldehyde and from 20 to 60% by weight of water; stream E1 comprises preferably from 0 to 1% by weight of trioxane, from 45 to 65% by weight of formaldehyde and from 34 to 55% by weight of water.

Stream D2 is preferably fed to the fourth distillation column as a side feed in the stripping section of the column. Stream F2 is likewise fed as a side feed. However, it is also possible to mix streams D2 and F2 and to feed them together as a side feed into the fourth distillation column.

At the top of the fourth distillation column, stream E1 is generally obtained and, in one embodiment, is fed to the trioxane synthesis reactor. In a further embodiment, stream E1 is fed to the formaldehyde concentration stage.

The stream E2 consisting substantially of water is obtained as a bottom draw stream or as a side draw stream in the stripping section of the column.

In addition to water, formaldehyde and trioxane, streams A2, B1, C1, D2, and E1 in particular may also comprise up to 15% by weight, generally from 1 to 10% by weight of low boilers. Typical low boilers which may be formed in the trioxane synthesis and the subsequent distillative separation are methyl formate, methylal, dimethoxydimethyl ether, trimethoxydimethyl ether, methanol, formic acid and also further hemiacetals and full acetals. To remove these low boilers, it is optionally possible after the first distillation stage b) to carry out a further distillation stage (low boiler removal stage). In this case, the low boilers are removed preferably via the top of the low boiler removal column which is preferably operated at a pressure of from 1 to 3 bar. In general, the low boiler removal column has at least 5 theoretical plates, preferably from 15 to 50 theoretical plates. The stripping section of this column comprises preferably from 25 to 90% of the theoretical plates of this column. Stream B1 is fed to this low boiler removal column as a side feed, and the stream B1′ freed of the low boilers is generally obtained as a bottom draw stream. When the low boiler removal is carried out, stream B1′ is fed as stream B1 to the downstream second distillation column. The recycled stream D1 can likewise also be fed to this low boiler column when this stream comprises low boilers.

The concentration e) of the feed stream F1 comprising water and formaldehyde is generally carried out in a distillation column or in an evaporator. The concentration is preferably carried out in an evaporator, more preferably in a continuous evaporator. Suitable continuous evaporators are, for example, circulation evaporators, falling-film evaporators, helical tube evaporators or thin-film evaporators. Particular preference is given to using falling-film evaporators to concentrate the water/formaldehyde mixture. The falling-film evaporator is operated generally at a pressure of from 50 to 200 mbar and a temperature of from 40 to 75° C.

The concentration step e) can be carried out as described, for example, in DE-A 199 25 870.

The formaldehyde-enriched stream A1 obtained in the concentration is generally withdrawn as a bottom draw stream; the low-formaldehyde stream F2 is withdrawn as a top or vapor draw stream.

When a distillation column is used for concentration, the feed stream F1 comprising water and formaldehyde is preferably fed as a side feed.

The pure trioxane obtained, whose purity may be >99% by weight, preferably >99.5% by weight or even >99.8% by weight, is preferably used to prepare polyoxymethylene (POM), polyoxymethylene derivatives such as polyoxy-methylene dimethyl ether (POMDME), and diaminodiphenylmethane (MDA).

The invention is described in detail below with reference to the drawing.

In the drawing:

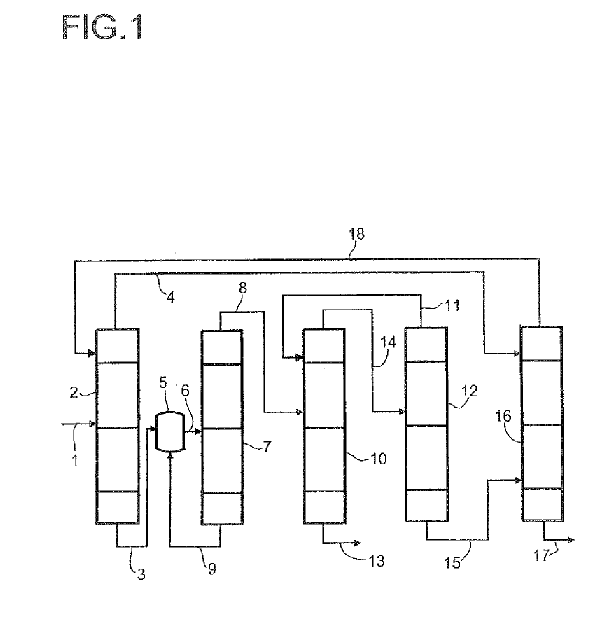

FIG. 1 shows a process flow diagram of a first variant of the process according to the invention,

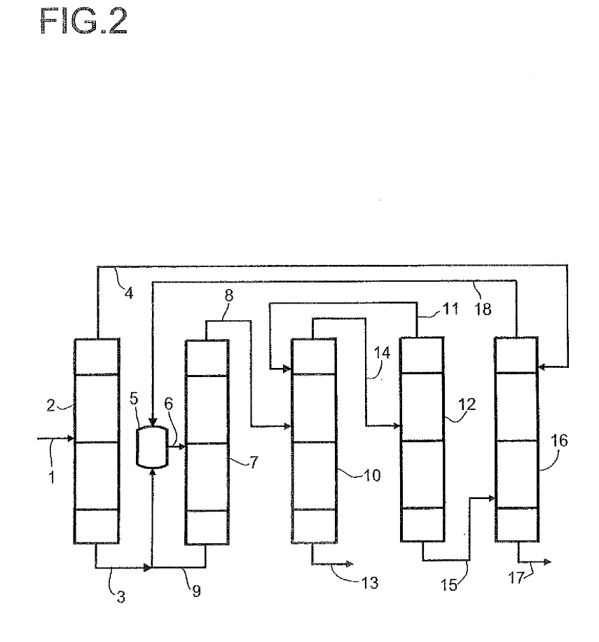

FIG. 2 shows a process flow diagram of a second variant of the process according to the invention.

FIG. 1 shows a first variant of the process according to the invention.

An aqueous formaldehyde solution 1 (stream F1) is added to a concentration unit 2. The concentration unit 2 may be any distillation column, for example a tray column, column with random packing or column with structured packing or a continuous evaporator, for example a circulation evaporator, falling-film evaporator, helical tube evaporator or thin-film evaporator. The concentration unit 2 is preferably a falling-film evaporator. From the concentration unit 2, a formaldehyde-rich bottom draw stream 3 (stream A1) and a low-formaldehyde aqueous vapor stream as top draw stream 4 (stream F2) are obtained. The formaldehyde-rich bottom draw stream 3 is fed to a trioxane synthesis reactor 5. In the trioxane synthesis reactor 5, the aqueous formaldehyde solution is reacted in the presence of an acidic catalyst present in homogeneous or heterogeneous form to give trioxane.

From the trioxane synthesis reactor 5, a stream 6 comprising trioxane, formaldehyde and water (stream A2) is fed as a side feed to a first distillation column 7. In the first distillation column 7, stream 6 is separated into a trioxane-enriched stream 8 (stream B1) which is withdrawn from the first distillation column 7 as a top draw stream and a stream 9 which is obtained as a top draw and consists substantially of water and formaldehyde (stream B2). The stream 9 obtained at the bottom (stream B2) is recycled into the trioxane synthesis reactor 5.

The stream 8 obtained at the top of the first distillation column 7 (stream B1) is fed to a second distillation column 10. In addition, a recycle stream 11 which is obtained at the top of a third distillation column 12 and comprises trioxane, water and formaldehyde is fed to the second distillation column 10. The streams 8 and 11 fed to the second distillation column 10 are separated into a product stream 13 comprising substantially trioxane (stream C2) and a stream 14 comprising trioxane, water and formaldehyde (stream C1) which is drawn off at the top of the second distillation column 10. Stream 14 is fed via a side feed to the third distillation column 12. In the third distillation column 12, stream 14 is separated into the recycle stream 11 which comprises trioxane, formaldehyde and water and is obtained at the top (stream D1) and a stream 15 consisting substantially of formaldehyde and water (stream D2) which is drawn off at the bottom of the third distillation column. Stream 15 (stream D2) is fed as a side feed to the stripping section of a fourth distillation column 16. Also fed to the fourth distillation column 16 is the top draw stream 4 (stream F2) of the concentration unit 2 as a side feed at the top. In the fourth distillation column 16, the streams 4, 15 fed are then separated into a stream 17 which comprises substantially water and is obtained at the bottom (stream E2) and a stream 18 which is obtained at the top of the fourth distillation column 16 and comprises formaldehyde and water (stream E1). Stream 18, which is substantially trioxane-free, is fed into the concentration unit 2.

FIG. 2 shows a second variant of the process according to the invention.

The process illustrated in FIG. 2 differs from the variant illustrated in FIG. 1 by the stream 18 obtained at the top of the fourth distillation column 16 not being conducted into the concentration unit 2 but rather into the trioxane synthesis reactor 5. The formaldehyde-rich bottom draw stream 3 of the concentration unit 2 and the stream 9 obtained at the bottom of the first distillation column 7 (stream B2) are also mixed before they are added to the synthesis reactor 5 and not added separately to the synthesis reactor 5.

### EXAMPLES

### Example 1

An aqueous formaldehyde solution 1 comprising 37% by weight of formaldehyde and 63% by weight of water is added to a concentration unit 2 designed as a falling-film evaporator. The falling-film evaporator is operated at a pressure of 100 mbar and a temperature of 50° C. A bottom draw stream 3 comprising 50% by weight of formaldehyde and 50% by weight of water is withdrawn from the bottom of the falling-film evaporator. The top draw stream 4 comprises 20% by weight of formaldehyde, the remainder is water.

The bottom draw stream 3 is fed to the trioxane synthesis reactor 5. The trioxane synthesis reactor is designed as a stirred tank reactor and is operated at a temperature of 108° C. The discharge stream 6 comprises 9% by weight of trioxane and 66% by weight of formaldehyde, the remainder is water.

The stream 6 is fed to a first distillation column 7 on the fifth tray. The first distillation column 7 is operated at a pressure of 1 bar. The temperature at the top is about 99° C., the temperature at the bottom is about 104° C. The first distillation column contains 24 trays. A stream 9 with a concentration of 80% by weight of formaldehyde and 20% by weight of water is withdrawn from the bottom of the first distillation column 7. A stream 8 with a concentration of 34% by weight of trioxane, 16% by weight of formaldehyde, and 50% by weight of water is withdrawn from the top of the first distillation column 7.

The stream 8 is fed to a second distillation column 10. The second distillation column 10 is operated at a pressure of 4 bar. The temperature at the top is about 142° C., the temperature at the bottom is about 166° C. The second distillation column has 40 trays, stream 8 is fed to the 20^{th} tray. Additionally, a recycle stream 11 from the process is fed to the second distillation column on the 30^{th} tray. The recycle stream 11 comprises 71% by weight of trioxane and 6% by weight of formaldehyde, the remainder is water. A stream 14 comprising 64% by weight of trioxane, 8% by weight of formaldehyde and 28% by weight of water is withdrawn from the top of the second distillation column 10. The product stream 13 comprising more than 99% by weight of trioxane is withdrawn from the bottom.

The top stream 14 is fed to a third distillation column 12 on the 24^{th} tray. The third distillation column 12 contains 48 trays and is operated at a pressure of 1 bar. The temperature at the top is about 101° C., the temperature at the bottom is about 104° C. The stream 15 is withdrawn from the bottom of the third distillation column. The stream 15 comprises 24% by weight of formaldehyde, the remainder is water. The top stream of the third distillation column is recycled to the second distillation column 10 as recycle stream 11.

The stream 15 is fed to a fourth distillation column 16 on the 24^{th} tray. Likewise, the top draw stream 4 of the falling-film evaporator is fed to the fourth distillation column 16 on this tray. The fourth distillation column 16 is operated at a pressure of 4 bar. The temperature at the top is about 137° C., the temperature at the bottom is about 145° C. The top stream 18 comprises 57% by weight of formaldehyde, the remainder is water. The bottom stream 17 comprises more than 98% by weight of water. The top stream 18 is recycled to the falling-film evaporator.

### Example 2

An aqueous formaldehyde solution 1 comprising 37% by weight of formaldehyde and 63% by weight of water is added to a concentration unit 2 designed as a falling-film evaporator. The falling-film evaporator is operated at a pressure of 100 mbar and a temperature of 50° C. A bottom draw stream 3 comprising 50% by weight of formaldehyde and 50% by weight of water is withdrawn from the bottom of the falling-film evaporator. The top draw stream 4 comprises 20% by weight of formaldehyde, the remainder is water.

The bottom draw stream 3 is fed to the trioxane synthesis reactor 5. The trioxane synthesis reactor is designed as a stirred tank reactor and is operated at a temperature of 108° C. The discharge stream 6 comprises 9% by weight of trioxane and 66% by weight of formaldehyde, the remainder is water.

The stream 6 is fed to a first distillation column 7 on the fifth tray. The first distillation column 7 is operated at a pressure of 1 bar. The temperature at the top is about 99° C., the temperature at the bottom is about 104° C. The first distillation column contains 24 trays. A stream 9 with a concentration of 80% by weight of formaldehyde and 20% by weight of water is withdrawn from the bottom of the first distillation column 7. A stream 8 with a concentration of 38% by weight of trioxane, 15% by weight of formaldehyde, and 47% by weight of water is withdrawn from the top of the first distillation column 7.

The stream 8 is fed to a second distillation column 10. The second distillation column 10 is operated at a pressure of 4 bar. The temperature at the top is about 142° C., the temperature at the bottom is about 166° C. The second distillation column has 40 trays, stream 8 is fed to the 20^{th} tray. Additionally, a recycle stream 11 from the process is fed to the second distillation column on the 30^{th} tray. The recycle stream 11 comprises 71% by weight of trioxane and 6% by weight of formaldehyde, the remainder is water. A stream 14 comprising 64% by weight of trioxane, 8% by weight of formaldehyde and 28% by weight of water is withdrawn from the top of the second distillation column 10. The product stream 13 comprising more than 99% by weight of trioxane is withdrawn from the bottom.

The top stream 14 is fed to a third distillation column 12 on the 24^{th} tray. The third distillation column comprises 48 trays and is operated at a pressure of 1 bar. The temperature at the top is about 101° C., the temperature at the bottom is about 104° C. The stream 15 is withdrawn from the bottom of this column. The stream 15 comprises 24% by weight of formaldehyde, the remainder is water. The top stream of the third distillation column 12 comprises 71% by weight of trioxane and 6% by weight of formaldehyde, the remainder is water. This stream is recycled to the second distillation column 10 as recycle stream 11.

The stream 15 is fed to a fourth distillation column 16 on the 24^{th} tray. Likewise, the top draw stream 4 of the falling-film evaporator is fed to the fourth distillation column 16 on this tray. The fourth distillation column 16 is operated at a pressure of 4 bar. The temperature at the top is about 137° C., the temperature at the bottom is about 145° C. The top stream 18 comprises 57% by weight of formaldehyde, the remainder is water. The bottom stream 17 comprises more than 98% by weight of water. The top stream 18 is recycled to the trioxane synthesis reactor 5.

1-15. (canceled)

16. An integrated process for preparing trioxane from formaldehyde, comprising:
a) feeding a stream A1 comprising water and formaldehyde and a recycle stream B2 comprising water and formaldehyde into a trioxane synthesis reactor, wherein the formaldehyde is converted to trioxane, to obtain a product stream A2 comprising trioxane, water, and formaldehyde;
b) feeding said stream A2 into a first distillation column and distilling at a pressure in the range of from 0.1 to 2.5 bar to obtain a stream B1 enriched in trioxane relative to the amount of trioxane in said stream A2 and a stream B2 comprising water and formaldehyde;
c) feeding said stream B1 and a recycle stream D1 comprising trioxane, water, and formaldehyde into a second distillation column and distilling at a pressure in the range of from 0.2 to 17.5 bar to obtain a product stream C2 comprising trioxane and a stream C1 comprising trioxane, water, and formaldehyde;
d) feeding said stream C1 into a third distillation column and distilling at a pressure in the range of from 0.1 to 2.5 bar to obtain said recycle stream D1 and a stream D2 comprising water and formaldehyde;
e) feeding a feed stream F1 comprising water and formaldehyde into a formaldehyde concentration unit, withdrawing said stream A1 from said formaldehyde concentration unit as a formaldehyde-rich bottom draw stream and withdrawing a low-formaldehyde stream F2 from said formaldehyde concentration unit as a top draw stream or a vapor draw stream;
f) feeding said streams D2 and F2 into a fourth distillation column and distilling at a pressure in the range of from 1 to 10 bar to obtain a stream E1 comprising water and formaldehyde and a stream E2 comprising water and feeding said stream E1 into said trioxane synthesis reactor or said formaldehyde concentration unit.

17. The process of claim 16, wherein the distillations in b) and d) are carried out at a pressure in the range of from 0.4 to 1.5 bar and the distillation in c) is carried out at a pressure in the range of from 2 to 7 bar.

18. The process of claim 16, wherein said stream B1 is withdrawn as a top draw stream and said stream B2 is withdrawn as a bottom draw stream from said first distillation column.

19. The process of claim 16, wherein said stream B1 is fed as a first side feed to said second distillation column, said stream D1 is fed as a second side feed to said second distillation column, said stream C1 is withdrawn as a top draw stream from said second distillation column, and said stream C2 is withdrawn as a bottom draw stream from said second distillation column.

20. The process of claim 16, wherein said streams B1 and D1 are mixed and then fed as a side feed to said second distillation column.

21. The process of claim 16, wherein said stream C1 is fed as a side feed to said third distillation column, said stream D1 is withdrawn as a top draw stream from said third distillation column, and said stream D2 is withdrawn as a bottom draw stream from said third distillation column.

22. The process of claim 16, wherein said stream D2 is fed as a first side feed to said fourth distillation column, said stream F2 is fed as a second side feed to said fourth distillation column, said stream E1 is withdrawn as a top draw stream from said fourth distillation column, and said stream E2 is withdrawn as a bottom draw stream from said fourth distillation column.

23. The process of claim 16, wherein said streams D2 and F2 are mixed and then fed as a side feed to said fourth distillation column.

24. The process of claim 16, wherein the distillation in f) is carried out at a pressure in the range of from 2 to 5 bar.

25. The process of claim 16, wherein said formaldehyde concentration unit is a distillation column.

26. The process of claim 16, wherein said formaldehyde concentration unit is an evaporator.

27. The process of claim 26, wherein said evaporator is a falling-film evaporator.

28. The process of claim 16, wherein said stream E1 is fed to said formaldehyde concentration unit.
